(19) United States
(12) Patent Application Publication
(10) Pub. No.: US 2010/0008396 A1
(43) Pub. Date: Jan. 14, 2010

(54) Method For Determining Internal Temperature of Meat Products

(76) Inventors: David Gaskins, Boaz, AL (US); Gregory Scott Spence, Jacksonville, AL (US); Allison Appleby, Southside, AL (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 12/172,652

(22) Filed: Jul. 14, 2008

(51) Int. Cl. G01K 13/00; G01N 33/12

(52) U.S. Cl. 374/141; 426/231

(57) ABSTRACT

Aspects of the present invention are directed to means and methods of the determination of the internal temperature of a meat product. Methods may comprise: a) providing the meat product on a conveying belt; b) compressing the meat product to induce liquid flow from the interior of the meat product toward a surface of the meat product; c) sensing an indication temperature of at least a portion of the surface of the meat product after compressing the meat product; and d) evaluating the indication temperature to determine whether the meat product is acceptable according to predetermined standards. Benefits of the present invention over the current art include the ability to determine the internal temperature of more than a sampling of meat products in a preparation process, a reduced risk of contamination, and a more accurate temperature reading.

### TECHNOLOGY FIELD

The present invention relates to temperature measurements, and, more particularly, to means and methods for determining the internal temperature of meat products.

### BACKGROUND

In the preparation of cooked meat products, it is important to ensure that all of the meat is indeed fully cooked. If a frozen product enters the meat preparation process, there is a likelihood that the product will be undercooked at the end of the process. Such undercooked meat may lead to various problems for consumers, including illness. Current practices to prevent such problems involve manually inserting stick probes into products as they exit the cooking process to ensure that the product is fully cooked.

There are several drawbacks to this process. First, because of the small sampling rate (about 1 out of every 100), the current practice is unlikely to identify an undercooked product exiting the meat preparation process. Second, temperature measured after the cooking process often fluctuates quickly and the temperature reading may not be a reliable indicator of internal temperature. Finally, precise placement of the probe is difficult and the temperature of the coolest portion of the product may not be taken.

### SUMMARY

Aspects of the present invention are directed to methods for determining the internal temperature of a meat product, comprising the steps of: a) providing the meat product on a conveying belt; b) compressing the meat product to induce liquid flow from the interior of the meat product toward a surface of the meat product; c) sensing an indication temperature of at least a portion of the surface of the meat product after compressing the meat product; and d) evaluating the indication temperature to determine whether the meat product is acceptable according to predetermined standards.

Evaluating the indication temperature may comprise comparing the indication temperature to a predetermined temperature. The predetermined temperature may be dependent upon the type of meat product. In certain embodiments, the predetermined temperature is less than about 35° F. In other embodiments, the predetermined temperature is less than about 32° F. Evaluating the indication temperature may comprise comparing the indication temperature to a temperature of an uncompressed portion of the meat product. In some aspects, the temperature of the uncompressed portion of the meat product is measured prior to compressing the meat product.

The step of compressing the meat product may be performed by a compression belt located above the meat product. Further, the compression belt may run parallel to the conveying belt at approximately the same speed of the conveying belt. The distance between the compression belt and the conveying belt upon completion of the compression step b) may be set to a predetermined distance. In some embodiments, the step of compressing the meat product is performed by a horn apparatus.

The step of sensing an indication temperature c) may include using an infrared thermal imaging device. The infrared thermal imaging device may be spaced apart and structurally isolated from the conveying belt and the compression belt or horn apparatus. The infrared thermal imaging device may be housed in a climate-controlled enclosure.

In certain embodiments, the meat product may be uncooked prior to the compression step b) and the temperature sensing step c). In other embodiments, the meat product may be marinated prior to providing the meat product on the moving belt. The meat product may comprise chicken breasts, chicken thighs, beef patties, beef products, pork products, poultry products, and like products.

In some embodiments, the meat product may be cooked before the temperature sensing of step c). In other embodiments, the meat product may be cooked after the temperature sensing of step c). Cooking may comprise passing the meat product through a belt grill, char marker, oven, or a combination thereof. In some embodiments, the thickness of the cooked meat products may be measured with a laser.

Some aspects of the present invention are related to a meat preparation apparatus comprising: a conveying belt adapted for conveying meat products; a press that moves at approximately the same linear speed as the conveying belt and is adapted for compressing at least a portion of a meat product; and at least one isolated thermal imaging device adapted for determining the temperature of a meat product. The apparatus may further comprise a cooking unit comprising a belt grill, a char marker, an oven, or a combination thereof. In some embodiments, the isolated thermal imaging device may be located before the cooking unit. In other embodiments, the isolated thermal imaging device may be located after the cooking unit. In still other embodiments, the isolated thermal imaging device is located before and after the cooking unit. The press in the apparatus may be a compression belt. In other embodiments, the press may be a horn apparatus.

### BRIEF DESCRIPTION OF THE DRAWINGS

The foregoing and other aspects of the present invention will become apparent from the following detailed description of the invention when considered in conjunction with the accompanying drawings. For the purpose of illustrating the invention, there is shown in the drawings embodiments that are presently preferred, it being understood, however, that the invention is not limited to the specific instrumentalities disclosed. In the drawings:

FIG. 1 is a side view schematic of an exemplary compression and non-contact temperature sensing process;

FIG. 2 is an elevational view of an exemplary meat preparation process using the non-contact temperature sensing process;

FIG. 3 is a flowchart of an exemplary meat preparation process using the non-contact temperature sensing process;

FIG. 4 is a flowchart of an exemplary cooking process;

FIG. 5 is a flowchart of an exemplary meat preparation process employing non-contact temperature sensing before and after the cooking process;

FIG. 6 is a flow chart of an exemplary cooking process employing non-contact temperature sensing after each step in the exemplary cooking process.

### DETAILED DESCRIPTION OF ILLUSTRATIVE EMBODIMENTS

Aspects of the present invention are related to methods to determine the internal temperature of a meat product during the meat preparation process. Certain embodiments of the present invention are directed to methods for determining the internal temperature of a meat product, comprising the steps of: a) providing the meat product on a conveying belt; b) compressing the meat product to induce liquid flow from the interior of the meat product toward a surface of the meat product; c) sensing an indication temperature of at least a portion of the surface of the meat product after compressing the meat product; and d) evaluating the indication temperature to determine whether the meat product is acceptable according to predetermined standards. The present invention possesses various improvements over the current state of the art. For example, the internal temperatures of more than a sampling of the meat products prepared during the meat preparation process may be determined. Further, the present invention does not require manual insertion of a temperature probe into meat products providing for a more accurate and safer way to determine the internal temperature of a meat product.

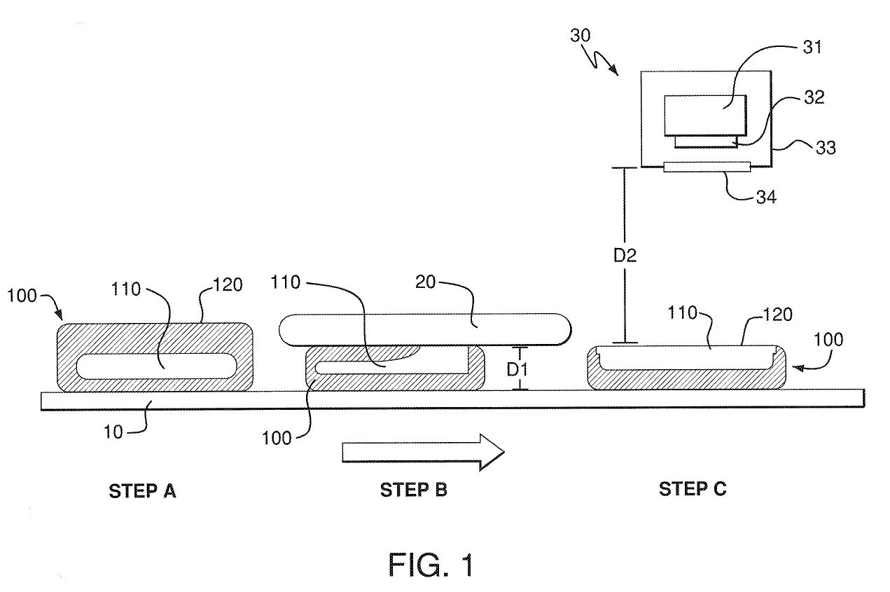

FIG. 1 shows the side view of one embodiment of the present invention exemplifying the compression of a meat product and the temperature sensing of the internal temperature of the meat product. As used herein, the term meat product may refer to any meat product suitable for preparation in a meat preparation process including, but not limited to chicken breasts, chicken thighs, beef patties, beef products, pork products, poultry products, and like products. Preferably, the meat product 100 is capable of being compressed to a relatively even thickness. The meat product 100 may be fully cooked, partially cooked, raw, frozen, partially frozen, thawed, or partially thawed.

In the embodiments shown in the figures, meat product 100 has an interior portion 110 and an exterior portion 120. Interior portion 110 of meat product 100 has a temperature that is less than exterior portion 120 of meat product 100. If the difference in temperature between interior portion 110 and exterior portion 120 is too large, the product may not be fully cooked after it passes through a cooking apparatus. The difference in temperature between interior portion 110 and the exterior portion 120 may vary depending upon factors such as thickness of the meat product, type of meat product, and state of the meat product (i.e. thawed, frozen, etc) among others.

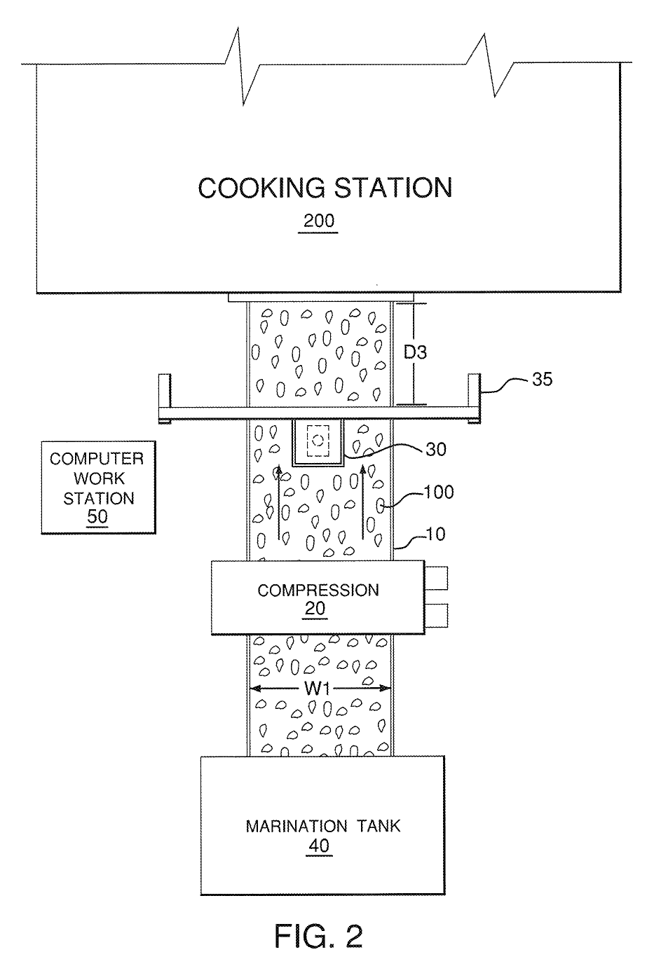

Meat product 100 may be provided on a conveying belt 10 (Step A). Suitable conveying belts are known to one of ordinary skill in the art. In certain embodiments, conveying belt 10 may have a width W1 (FIG. 2) of between about 30 inches and about 100 inches, preferably between about 36 inches and about 40 inches. The speed of conveying belt 10 may vary. In some embodiments, the speed of conveying belt 10 may be between about 5 feet/minute and about 50 feet/minutes, preferably between about 10 feet/minute and about 20 feet/minute.

Meat product 100 may be compressed 20 by a device above conveying belt 10 to produce a resultant meat product with a pre-determined thickness D1. In certain embodiments, the thickness of meat product 100 may be between about 2 mm and about 20 mm. In other embodiments, the thickness of meat product 100 may be between about 8 mm and about 15 mm. In still other embodiments, the thickness of meat product 100 may be between about 10 mm and about 13 mm.

Meat product 100 may be compressed by a press. The present invention has been illustrated with a compression belt to compress the meat product, but other presses may be utilized. For example, suitable presses may include one or more rams, one or more plates, one or more rollers on a fixed shaft, or one or more horns, among others. For presses that are moving, for example, a compression belt, the presses may move at approximately the same linear speed as conveying belt 10. In other embodiments, a horn is utilized to compress the meat product to a uniform thickness D1. A horn may provide for both vertical uniformity of the meat product and horizontal uniformity of the meat product. Suitable amounts of pressure applied by the press may vary depending upon the meat product being compressed, and the desired thickness of the meat product, among other variables.

Compression 20 of meat product 100 may induce liquid flow from interior portion 110 to exterior portion 120 (Step B). If meat product 100 is frozen or dry, liquid may not flow from interior portion 110 to exterior portion 120. It is intended, however, that the scope of claims directed to inducing liquid flow encompass frozen or dry meat products, wherein fluid flow does not actually occur, as well as meat products wherein fluid flow does occur.

After compression 20 of meat product 100, at least a portion of the liquid from interior portion 110 may move to at least a portion of exterior portion 120 (Step C). The liquid from interior portion 110, now present in the exterior portion 120 may have a temperature referred to as an indication temperature. The indication temperature may be evaluated to determine whether meat product 100 is suitable for proceeding along the preparation process. In certain embodiments, the indication temperature may be evaluated by comparing the indication temperature to a predetermined temperature selected depending upon factors such as the type of meat product, the state of the meat product, and the thickness of the meat product, among others. The predetermined temperature may range from between about 25° F. and about 125° F. In certain embodiments, the predetermined temperature may be between about 25° F. and about 35° F. In other embodiments, the predetermined temperature may be between about 40° F. and about 50° F. In other embodiments, the predetermined temperature may be between about 55° F. and about 65° F.

The indication temperature may be determined by a temperature sensing station 30 comprising a temperature sensing device 31 housed within an enclosure 33. Suitable temperature sensing devices may include an infrared thermal imaging device. Preferably, temperature sensing device 31 is an infrared thermal imaging device. Temperature sensing device 31 may further comprise a lens 32 suitable to image product across the entire width of conveying belt 10. The ability to image the entire width of conveying belt 10 depends on factors including width of the conveying belt W1, the height of the camera from the conveying belt D2 and the field of view of lens 32.

Enclosure 33 may further comprise a window 34 at the base of the enclosure to provide for visualization of meat product 100 by temperature sensing device 31 through window 34. In certain embodiments, enclosure 33 is climate controlled. Climate control within enclosure 33 provides for a more stable atmosphere within enclosure 33 and helps to eliminate any variations in the performance of temperature sensing device 31 due to external environment changes. Enclosure 33 may be supported by support beams 35 to position temperature sensing device 31 above conveying belt 10. Preferably, support beams 35 are not in physical contact with conveying belt 10 or any other component of the meat preparation process other than enclosure 33 and temperature sensing device 31. Such physical isolation reduces the effect of vibration associated with meat preparation process on temperature sensing device 31.

Temperature sensing device 31 may be positioned at a distance D2 from conveying belt 10 wherein the entire width W1 of conveying belt 10 may be evaluated by temperature sensing device 31. The distance between temperature sensing device 31 and conveying belt 10 is dependent upon various factors, for example, width of the conveying belt W1 and the field of view of lens 32 attached to the temperature sensing device 31. In certain embodiments, D2 may be between about 40 and about 100 inches or, for example, between about 50 and about 90 inches or, for example, between about 60 and about 80 inches or, for example, between about 70 and about 80 inches. The field of view of the camera may vary depending upon conveying belt width W1 and the distance between the temperature sensing device and the conveying belt D2. In some embodiments, the field of view of lens 32 is between about 15 degrees and about 35 degrees, preferably between about 20 degrees and about 30 degrees, more preferably between about 24 degrees and 28 degrees.

Temperature sensing device 31 may be controlled by a computer work station 50. Computer work station 50 may be utilized to input values such as a suitable indication temperature, speed of the conveying belt, and various values related to the temperature sensing device, among others. Computer work station 50 may also be utilized to sound an alarm when an indication temperature falls out of a suitable indication temperature range. Further, computer work station 50 may be programmed to shut down conveying belt 10, or to slow down conveying belt 10 to allow meat product 100 to reach a suitable indication temperature.

In certain embodiments, the external temperature of meat product 100 may be taken prior to compression of meat product 100. The external temperature may be taken using a device known to one skilled in the art. In certain embodiments, the external temperature of the uncompressed portion may be compared with the indication temperature to determine whether the meat product is suitable to proceed along the preparation process. This comparison may be performed by computer work station 50.

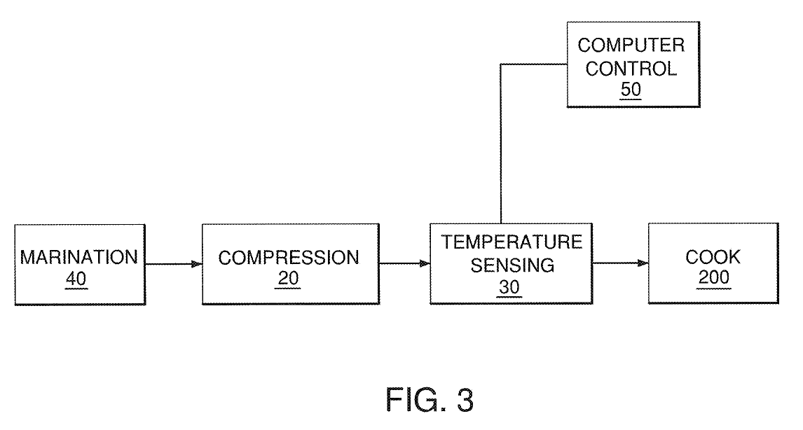

In certain embodiments of the present invention, meat product 100 may be marinated 40 prior to compression 20 as seen in FIG. 2 and FIG. 3. The marination step may be performed in a device known to one skilled in the art and may comprise a tank or a tumbler. The marination step may be performed prior to meat product 100 placement onto conveying belt 10 or it may be performed while meat product 100 is on conveying belt 10. For example, conveying belt 10 may pass through a marination tank. Preferably, marination 40 is performed in a tumbler prior to placement of meat product 100 onto conveying belt 10.

Meat product 100 may also be passed through a cooking station 200 while on conveying belt 10. Cooking station 200 may be after the temperature sensing of meat product 100. In certain embodiments, the distance D3 between temperature sensing station 30 and cooking station 200 may be controlled to limit the risk of heat from cooking station 200 affecting the results from temperature sensing station 30. For example, distance D3 between temperature sensing station 30 and cooking station 200 may be between about 2 feet and about 6 feet, preferably between about 4 feet and about 5 feet.

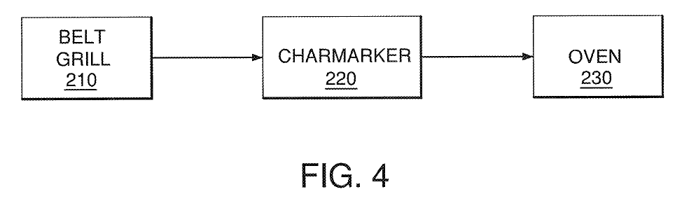

FIG. 4 shows one embodiment of exemplary steps within the cooking station. For example, cooking station 200 may comprise a belt grill 210, a charmarker, 220, an oven 230, or any combination thereof depending upon the needs of the user. Cooking station 200 may be operated at various temperatures depending upon the application. In some embodiments, the temperature within the cooking station is between about 100° F. and about 200° F, preferably between about 110° F. and about 150° F.

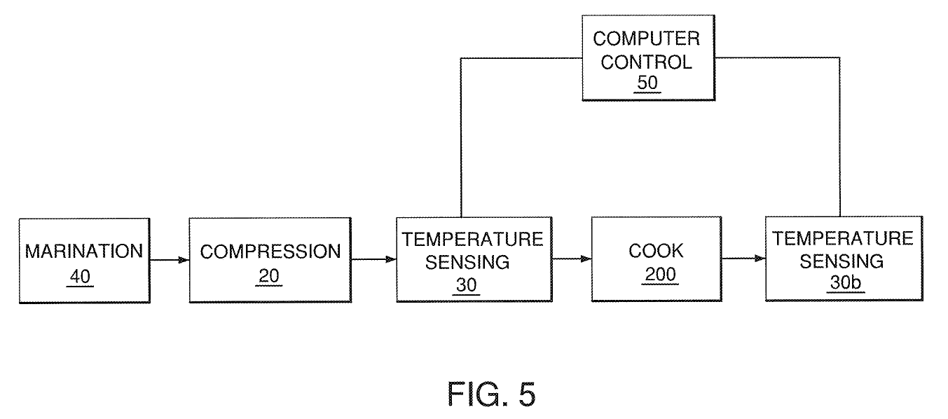

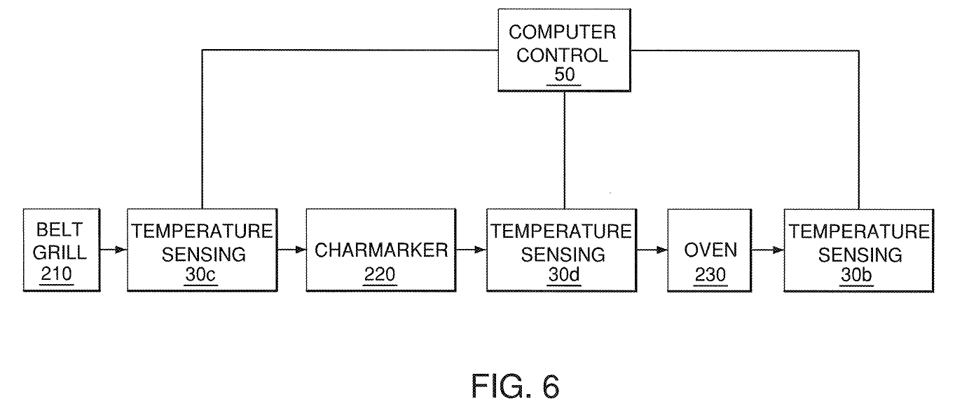

In certain embodiments, the present invention may comprise multiple locations wherein temperature sensing may be performed. For example, temperature sensing stations may be located both before 30 and after 30b the cooking process 200. (FIG. 5) In some embodiments, the multiple temperature sensing devices 31 are in contact with a single computer work station 50. In one embodiment, an additional temperature sensing station 30 may be positioning at the exit of cooking station 200 to determine the indication temperature of meat product 100. Temperature sensing may also be performed after individual steps within the cooking process. FIG. 6 shows one exemplary embodiment wherein temperature sensing is employed after meat product 100 is passed through belt grill 210, after the meat product is passed through char marker 220, and after the meat product is passed through oven 230. In some embodiments, temperature sensing devices 31 may be controlled by separate computer work stations 50 or they may be controlled by a single computer work station 50. In embodiments wherein the indication temperature of the meat product is determined after the cooking process or at least one step within the cooking process, an alternative technique may be employed to determine the thickness of the meat product. For example, a laser may be utilized to determine thickness.

What is claimed:

1. A method for determining the internal temperature of a meat product, comprising the steps of:
a) providing the meat product on a conveying belt;
b) compressing the meat product to induce liquid flow from the interior of the meat product toward a surface of the meat product;
c) sensing an indication temperature of at least a portion of the surface of the meat product after compressing the meat product; and
d) evaluating the indication temperature to determine whether the meat product is acceptable according to predetermined standards.

2. The method of claim 1 wherein evaluating the indication temperature comprises comparing the indication temperature to a predetermined temperature.

3. The method of claim 2 wherein the predetermined temperature is dependent upon the type of meat product.

4. The method of claim 2 wherein the predetermined temperature is less than about 35° F.

5. The method of claim 4 wherein the predetermined temperature is less than about 32° F.

6. The method of claim 1 wherein evaluating the indication temperature comprises comparing the indication temperature to a temperature of an uncompressed portion of the meat product.

7. The method of claim 6 wherein the temperature of the uncompressed portion of the meat product is measured prior to compressing the meat product.

8. The method of claim 1 wherein the step of compressing the meat product is performed by a compression belt located above the meat product.

9. The method of claim 8 wherein the compression belt runs parallel to the conveying belt at approximately the same speed of the conveying belt.

10. The method of claim 8 wherein the distance between the compression belt and the conveying belt upon completion of the compression step b) is set to a predetermined distance.

11. The method of claim 1 wherein the step of compressing the meat product is performed by a horn apparatus wherein the compression occurs in both the horizontal and vertical direction.

12. The method of claim 1 wherein the step of sensing an indication temperature c) includes using an infrared thermal imaging device.

13. The method of claim 12 wherein the infrared thermal imaging device is spaced apart and structurally isolated from the conveying belt and the compression belt or horn apparatus.

14. The method of claim 13 wherein the infrared thermal imaging device is housed in a climate-controlled enclosure.

15. The method of claim 1 wherein the meat product is uncooked prior to the compression step b) and the temperature sensing step c).

16. The method of claim 1 further comprising marinating the meat product prior to providing the meat product on the moving belt.

17. The method of claim 1 wherein the meat product comprises chicken breasts, chicken thighs, beef patties, beef products, pork products, or poultry products.

18. The method of claim 1 further comprising cooking the meat product before the temperature sensing of step c).

19. The method of claim 1 further comprising cooking the meat product after the temperature sensing of step c).

20. The method of claim 19 wherein cooking comprises passing the meat product through a belt grill, char marker, oven, or a combination thereof.

21. The method of claim 19 further comprising measuring the thickness of the cooked meat products with a laser.

22. A meat preparation apparatus comprising:
a) A conveying belt adapted for conveying meat products;
b) A press that moves at approximately the same linear speed as the conveying belt and is adapted for compressing at least a portion of a meat product; and
c) At least one isolated thermal imaging device adapted for determining the temperature of a meat product.

23. The apparatus of claim 22 further comprising a cooking unit comprising a belt grill, a char marker, an oven, or a combination thereof.

24. The apparatus of claim 22 wherein the isolated thermal imaging device is located before the cooking unit.

25. The apparatus of claim 22 wherein the isolated thermal imaging device is located after the cooking unit.

26. The apparatus of claim 22 wherein the isolated thermal imaging device is located before and after the cooking unit.

27. The apparatus of claim 22 wherein the press is a compression belt.

28. The apparatus of claim 22 wherein the press is a horn apparatus.
